(19) United States
(12) Patent Application Publication
(10) Pub. No.: US 2022/0132948 A1
(43) Pub. Date: May 5, 2022

(54) SCENTED AROMA INTEGRATED INTO POLYMER FIBERS OF MASK MEDIA

(71) Applicant: CUMMINS FILTRATION INC., Nashville, TN (US)

(72) Inventors: Mahsa Kasiri, Fitchburg, WI (US); Corinne Muha, Mount Juliet, TN (US); Cindy Lee Cotton, Indianapolis, IN (US); Elizabeth D. Mace, Seymour, IN (US); Wassem Abdalla, Fishers, IN (US); Gregory W. Hoverson, Nashville, TN (US); Eric A. Janikowski, Jefferson, WI (US); Ismail Bagci, Cookeville, TN (US)

(73) Assignee: CUMMINS FILTRATION INC., Nashville, TN (US)

(21) Appl. No.: 17/514,116

(22) Filed: Oct. 29, 2021

Related U.S. Application Data
(60) Provisional application No. 63/109,649, filed on Nov. 4, 2020.

(51) Int. Cl. A41D 13/11; A62B 18/02; A61L 9/013; D01D 5/08

(52) U.S. Cl. CPC A41D 13/11; A62B 18/025; A61L 2209/133; D01D 5/08; A61L 9/013

(57) ABSTRACT

A face mask includes an aroma layer. The aroma layer includes one or more melt-blown layers or one or more scrim layers. The aroma layer includes a plurality of fibers with an aroma integrated therewith. The aroma layer includes an aroma layer outer side and an aroma layer inner side. The face mask includes an outer scrim layer and an inner scrim layer. The outer scrim layer is positioned adjacent the aroma layer outer side. The inner scrim layer is positioned adjacent the aroma layer inner side.

### CROSS-REFERENCE TO RELATED APPLICATIONS

The present application claims the benefit of U.S. Provisional Application No. 63/109,649, filed Nov. 4, 2020, the entire contents of which are incorporated herein by reference.

### TECHNICAL FIELD

The present invention relates generally to face masks.

### BACKGROUND

Face masks are worn by individuals to protect wearers and those in proximity to the wearer. Face masks can reduce or prevent the transmission of infectious particles exiting the wearer's nose and mouth. When the wearer breathes, coughs, or speaks, the face mask can act as a physical barrier and filter out the viral particles and bacteria from the air exiting the face mask. Face masks can also prevent the transmission of infectious particles from the outside environment to the wearer. However, when a face mask is worn by an individual for an extended period of time, the air within the face mask can become stagnant.

### SUMMARY

According to one embodiment, a face mask is provided. The face mask includes an aroma layer. The aroma layer includes one or more melt-blown layers. The one or more melt-blown layers include a plurality of fibers with an aroma integrated therewith.

According to another embodiment, the face mask includes an aroma layer. The aroma layer includes one or more scrim layers. The one or more scrim layers include a plurality of fibers with an aroma integrated therewith.

According to another embodiment, the face mask includes an aroma layer. The aroma layer includes an aroma layer outer side and an aroma layer inner side. The face mask includes an outer scrim layer positioned adjacent the aroma layer outer side and an inner scrim layer positioned adjacent the aroma layer inner side.

According to another embodiment, the face mask includes a middle layer. The middle layer includes one or more scrim layers and one or more melt-blown layers. The middle layer includes a middle layer outer side and a middle layer inner side. The face mask includes an outer scrim layer positioned adjacent the middle layer outer side. The face mask includes an inner scrim layer positioned adjacent the middle layer inner side. At least one of the middle layer, the outer scrim layer, or the inner scrim layer includes a plurality of fibers with an aroma integrated therewith.

According to another embodiment, a method of manufacturing a face mask is provided. The method includes providing a blended mixture including polypropylene, a charge additive, and an aroma. The method includes extruding the blended mixture to form melt-blown fibers. The method includes forming an aroma layer from the melt-blown fibers. The method includes disposing the aroma layer between an outer mask layer and an inner mask layer. The method includes securing the outer mask layer and the inner mask layer.

According to another embodiment, a method of manufacturing a face mask is provided. The method includes providing the face mask and adding aroma to the face mask. These and other features, together with the organization and manner of operation thereof, will become apparent from the following detailed description when taken in conjunction with the accompanying drawings, wherein like elements have like numerals throughout the several drawings described below.

### BRIEF DESCRIPTION OF THE DRAWINGS

The foregoing and other features of the present disclosure will become more fully apparent from the following description and appended claims, taken in conjunction with the accompanying drawings. Understanding that these drawings depict only several implementations in accordance with the disclosure and are therefore, not to be considered limiting of its scope, the disclosure will be described with additional specificity and detail through use of the accompanying drawings.

FIG. 1 is an exploded perspective view of layers of a face mask, according to an embodiment.

FIG. 2 is a scanning electron microscope image of fibers of a face mask, according to an embodiment.

FIG. 3 is a schematic diagram for a method of manufacturing a face mask, according to an embodiment.

FIG. 4 is a schematic diagram for a method of manufacturing a face mask, according to an embodiment.

Reference is made to the accompanying drawings throughout the following detailed description. In the drawings, similar symbols typically identify similar components, unless context dictates otherwise. The illustrative implementations described in the detailed description, drawings, and claims are not meant to be limiting. Other implementations may be utilized, and other changes may be made, without departing from the spirit or scope of the subject matter presented here. It will be readily understood that the aspects of the present disclosure, as generally described herein, and illustrated in the figures, can be arranged, substituted, combined, and designed in a wide variety of different configurations, all of which are explicitly contemplated and made part of this disclosure.

### DETAILED DESCRIPTION

Embodiments described herein relate generally to face masks containing an aroma layer that includes a plurality of fibers with an aroma integrated therewith. The aroma layer can provide a scent to the wearer of the face mask, thereby preventing the air within the face mask from becoming stagnant. The aroma layer can increase the comfort of a wearer for the duration that the wearer wears the face mask. The aroma layer can include an aroma (e.g., aroma additive) integrated into melt-blown fibers or scrim fibers.

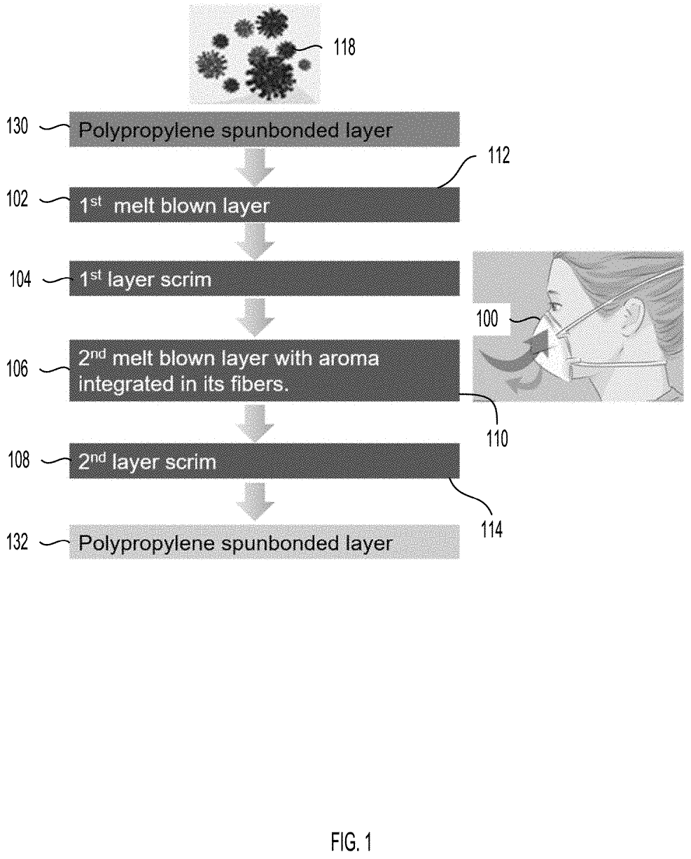

Referring to FIG. 1, an exploded view of layers of a face mask 100 (e.g., N95 mask, surgical mask) is shown, according to an embodiment. The face mask 100 and the wearer's face form an internal cavity when the wearer dons the face mask 100. When the wearer inhales, air passes through and is filtered by the layers of the face mask 100 as the internal cavity fills with incoming air. When the wearer exhales, the exhaled air passes through the internal cavity and the layers of the face mask 100 into the outside environment.

The face mask 100 can include an aroma layer 110. The aroma layer 110 can include one or more melt-blown layers. The one or more melt-blown layers include a plurality of fibers with an aroma integrated therewith. The one or more melt-blown layers can include a first melt-blown layer 102. The first melt-blown layer 102 can include a plurality of fibers with an aroma integrated therewith. The one or more melt-blown layers can include a second melt-blown layer 106. The second melt-blown layer 106 can include a plurality of fibers with an aroma integrated therewith. The plurality of fibers can include an anti-fog additive. The anti-fog additive can lower exhaled moisture and lower breathing resistance. The anti-fog additive can be added to or integrated into the plurality of fibers. The one or more melt-blown layers may be formed from a melt blowing fabrication process. The one or more melt-blown layers can include melt-blown polypropylene. The one or more melt-blown layers can include a filter for the face mask 100. The one or more melt-blown layers can prevent or reduce particles 118 (e.g., infectious particles, infectious droplets, bacteria, viral particles, aerosols, particulates) from penetrating through the rest of the face mask 100. The one or more melt-blown layers can prevent or reduce particles 118 from exiting the internal cavity. The particles 118 can include particles external to the wearer (e.g., particles from the outside the face mask 100). The particles 118 can include particles from the wearer (e.g., particles from the wearer and inside the internal cavity). The aroma layer 110 can include, for example, menthol, lavender, banana, apple, or Aloe vera.

The aroma layer 110 can include an aroma layer outer side 112. The face mask 100 can include an outer scrim layer 130. The outer scrim layer 130 can be positioned adjacent the aroma layer outer side 112. The outer scrim layer 130 can include a spunbonded layer (e.g., polypropylene spunbonded layer, scrim layer, spunbound layer, polypropylene spunbound layer etc.). The outer scrim layer 130 can serve as the first layer to prevent or reduce particles 118 from penetrating through the rest of the face mask 100. The outer scrim layer 130 can serve as the final layer to prevent or reduce particles 118 from exiting the internal cavity. The first melt-blown layer 102 can be positioned adjacent the outer scrim layer 130. The outer scrim layer 130 can be hydrophobic and prevent moisture from entering into the internal cavity. The outer scrim layer 130 can be splash-resistant. The outer scrim layer 130 can include, for example, menthol, lavender, banana, apple, or Aloe vera.

The aroma layer 110 can include an aroma layer inner side 114. The face mask 100 can include an inner scrim layer 132. The inner scrim layer 132 can be positioned adjacent the aroma layer inner side 114. The inner scrim layer 132 can include a spunbonded layer (e.g., polypropylene spunbonded layer, scrim layer, etc.). The inner scrim layer 132 can serve as the final layer to prevent or reduce particles 118 from penetrating through the rest of the face mask 100. The inner scrim layer 132 can serve as the first layer to prevent or reduce particles 118 from exiting the internal cavity. The inner scrim layer 132 can be directly exposed to the wearer's face. The inner scrim layer 132 include a texture that is comfortable when the inner scrim layer 132 is positioned adjacent to the wearer's face. The second melt-blown layer 106 can be positioned adjacent the inner scrim layer 132. The inner scrim layer 132 can include, for example, menthol, lavender, banana, apple, or Aloe vera.

The aroma layer 110 can include one or more scrim layers. The one or more scrim layers can include a first scrim layer 104. The one or more scrim layers can include a second scrim layer 108. The first scrim layer 104 can be positioned between the first melt-blown layer 102 and the second melt-blown layer 106. The second scrim layer 108 can be positioned between the second melt-blown layer 106 and the inner scrim layer 132. The one or more scrim layers can provide reinforcement and structural support to the face mask 100. The one or more scrim layers can prevent or reduce particles 118 from penetrating through the rest of the face mask 100. The one or more scrim layers can prevent or reduce particles 118 from exiting the internal cavity. The fibers of the one or more scrim layers can include course fibers, compared with the fine fibers of the one or more melt-blown layers.

In another embodiment, the aroma layer 110 includes one or more scrim layers. The aroma layer 110 can include a plurality of fibers with an aroma integrated therewith. The one or more scrim layers can include the first scrim layer 104. The first scrim layer 104 can include a plurality of fibers with an aroma integrated therewith. The one or more scrim layers can include the second scrim layer 108. The second scrim layer 108 can include a plurality of fibers with an aroma integrated therewith. The plurality of fibers can include the anti-fog additive. The anti-fog additive can lower exhaled moisture and breathing resistance. The one or more scrim layers can prevent or reduce particles 118 from penetrating through the rest of the face mask 100. The one or more scrim layers can prevent or reduce particles 118 from exiting the internal cavity. The outer scrim layer 130 can be positioned adjacent the aroma layer outer side 112. The first scrim layer 104 can be positioned adjacent the outer scrim layer 130. The inner scrim layer 132 can be positioned adjacent the aroma layer inner side 114. The second scrim layer 108 can be positioned adjacent the inner scrim layer 132.

In another embodiment, the face mask 100 includes a middle layer (e.g., aroma layer). The middle layer includes one or more scrim layers and one or more melt-blown layers. The middle layer includes a middle layer outer side and a middle layer inner side. The face mask 100 includes the outer scrim layer 130 positioned adjacent the middle layer outer side. The face mask 100 includes the inner scrim layer 132 positioned adjacent the middle layer inner side. At least one of the middle layer, the outer scrim layer 130, or the inner scrim layer 132 includes a plurality of fibers with an aroma integrated therewith. The middle layer can include, for example, menthol, lavender, banana, apple, or Aloe vera.

The face mask 100 can prevent particles 118 from penetrating through the layers of the face mask 100. The face mask 100 can include the aroma layer 110. The aroma layer 110 can be directly or indirectly exposed to the wearer's face. The layers of the face mask 100 can include the outer scrim layer 130, the first melt-blown layer 102, the first scrim layer 104, the second melt-blown layer 106, the second scrim layer 108, and the inner scrim layer 132. The layers of the face mask 100 can include the outer scrim layer 130, the first melt-blown layer 102, the first scrim layer 104, and the inner scrim layer 132. The first melt-blown layer 102 can include one or more melt-blown sublayers. For example, the first melt-blown layer 102 can include a first melt-blown sublayer, a second melt-blown sublayer, and a third melt-blown sublayer. The first scrim layer 104 can include one or more scrim sublayers. For example, the first scrim layer 104 can include a first scrim sublayer, a second scrim sublayer, and a third scrim sublayer. The face mask 100 can include Aloe vera. The Aloe vera can provide comfort to the wearer (e.g., user) when the wearer wears the face mask 100. The Aloe vera can treat, exfoliate, restore, and provide nutrition to the human skin. The aroma layer 110 can include Aloe vera.

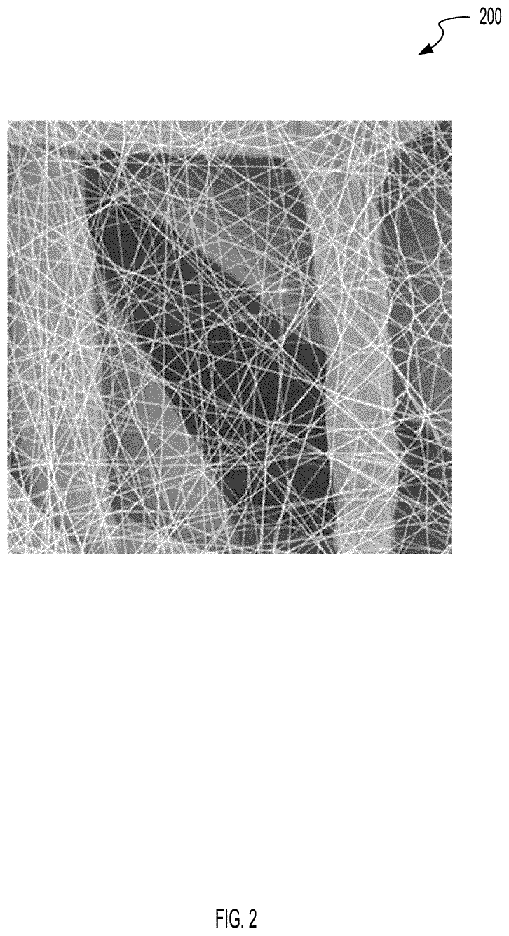

FIG. 2 is a scanning electron microscope (SEM) image 200 of fibers of a face mask, according to an embodiment. The aroma can be integrated in the plurality of fibers. The aroma integrated with the plurality of fibers produces a longer-lasting scent than aroma that is not integrated with the plurality of fibers. In some embodiments, the plurality of fibers include melt-blown polypropylene as an example media (e.g., mask media). The aroma can include various scents, scented aromas, fragrances, or oils. For example, the aroma can include, for example, menthol, which provides a cooling sensation to the wearer's face. The menthol can help the wearer's nasal passages open or stay open. The menthol can aid in improvement of the wearer's skin. For example, the menthol can prevent breakouts and provide moisture to the wearer's skin. The menthol can improve the wearer's breathing. The aroma can include fragrances such as lavender, apple, banana, sporty, or other customized scents. The aroma can be combined with additives. The aroma can reduce the effects and/or orders of bacteria. The aroma can be antimicrobial. The aroma can have a calming scent and increase the wearer's comfort. The aroma can control asthma symptoms by managing tension. The aroma can be added to the raw materials used to make the plurality of fibers. The aroma can be added to or integrated into media fibers. The aroma additive in the form of polypropylene resin can allow for a more beneficial manufacturing method, where scent cannot be damaged as in situations in which the aroma is in a more delicate form (e.g., sachet, gel, etc.).

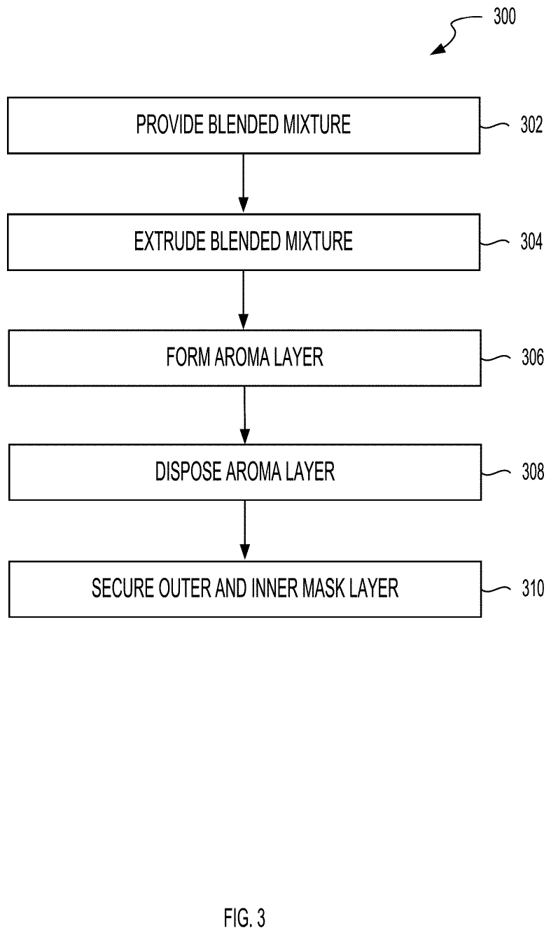

FIG. 3 shows a method 300 of manufacturing a face mask, according to an embodiment. As described further herein, at 302, a blended mixture is provided. At 304, the blended mixture is extruded. At 306, an aroma layer is formed. At 308, the aroma layer is disposed in the face mask. At 310, an outer mask layer and an inner mask layer are secured. The present method can produce a face mask of increased comfort and wearability as compared with conventional face masks.

At 302, a blended mixture (e.g., media, polymer melt) including polypropylene, a charge additive, and an aroma is provided. The charge additive can promote the charging of the media. The charge additive can allow the media to maintain or retain its charge. The blended mixture can include polypropylene in a range of 94 wt % to 98 wt % (e.g., 94 wt %, 95 wt %, 96 wt %, 97 wt %, or 98 wt %, inclusive). The blended mixture can include charge additive in a range of 2 wt % to 6 wt % (e.g., 2 wt %, 3 wt %, 3 wt %, 5 wt %, or 6 wt %, inclusive). The blended mixture can include trace amounts (e.g. less than 1 wt %) of aroma. The blended mixture can include the polypropylene, the charge additive, the aroma, and the anti-fog additive.

At 304, the blended mixture is extruded to form melt-blown fibers. For example, the blended mixture can be forced through a die to produce components of a fixed cross-sectional area. The blended mixture can include molten polymer. The aroma can be integrated with the blended mixture. The aroma can be integrated with the one or more melt-blown layers during the extrusion process. The aroma can be added to the one or more melt-blown layers.

At 306, an aroma layer is formed from the melt-blown fibers. The blended mixture can be formed into the aroma layer through a melt blowing process. The aroma layer can include a nonwoven sheet or melt-blown layer. The blended mixture can be extruded through nozzles surrounded by high speed blowing gas. Fibers (e.g., microfibers, nanofibers, polymer fibers) of the blended mixture can be deposited randomly to form the nonwoven sheet.

At 308, the aroma layer is disposed between an outer mask layer and an inner mask layer. The outer mask layer can include an outer scrim layer. The inner mask layer can include an inner scrim layer. The aroma layer is indirectly exposed to the wearer's face. For example, the aroma layer can be integrated into a layer than is not directly exposed to the wearer's face. In some embodiments, the aroma layer is disposed as the inner mask layer such that the aroma layer is directly exposed to the wearer's face. For example, the aroma layer can be integrated into a layer than is directly exposed to the wearer's face.

At 310, the outer mask layer and the inner mask layer are secured. The outer mask layer and the inner mask layer are secured such that the face mask forms a singular unit. In some embodiments, the method 300 can include blending polypropylene, the charge additive, and the aroma to form the blended mixture.

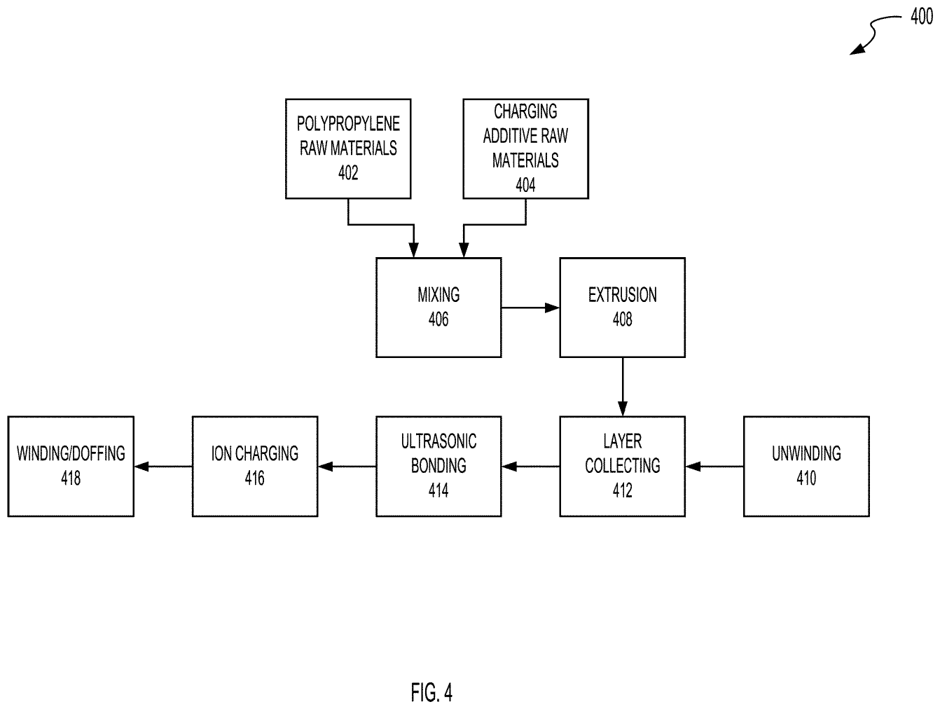

FIG. 4 shows a method 400 of manufacturing a face mask, according to another embodiment. The aroma can be added at various points in the manufacturing process. For example, the aroma can be added to polypropylene raw materials 402. The aroma can be integrated with the polypropylene raw materials. The aroma can be added to charging additive raw materials 404 (e.g., charge additive raw materials). The aroma can be integrated with the charging additive raw materials.

At 406, the polypropylene raw materials 402 can be mixed (e.g., combined) with the charging additive raw materials 404 to form a blended mixture. The polypropylene raw materials 402 can be mixed with the charging additive raw materials 404 in a mixing chamber. The aroma can be added to the mixing chamber. The blended mixture can include charge additive in a range of 2 wt % to 6 wt % (e.g., 2 wt %, 3 wt %, 3 wt %, 5 wt %, or 6 wt %, inclusive). The aroma can be integrated with the polypropylene raw materials and the charging additive raw materials during the mixing process.

At 408, the blended mixture can be extruded via a melt blowing process to form one or more melt-blown layers. For example, the blended mixture can be forced through a die to produce components of a fixed cross-sectional area. The blended mixture can include molten polymer. The aroma can be added to the one or more melt-blown layers. The aroma can be integrated with the blended mixture. The aroma can be integrated with the one or more melt-blown layers during the extrusion process.

At 410, one or more scrim layers can be unwound. For example, the one or more scrim layers can undergo an unwinding process. The one or more scrim layers can be separated. The aroma can be added to the one or more scrim layers. The aroma can be integrated with the one or more scrim layers during the unwinding process.

At 412, the one or more melt-blown layers can be combined with the one or more scrim layers. For example, the one or more melt-blown layers can be placed on the one or more scrim layers. The one or more scrim layers can be place on the one or more melt-blown layers. The one or more melt-blown layers can be positioned next to the one or more scrim layers. The one or more melt-blown layers can be interleaved with the one or more scrim layers. The one or more melt-blown layers can be stacked between the one or more scrim layers. The one or more scrim layers can be stacked between the one or more melt-blown layers. The aroma can be integrated with the one or more melt-blown layers. The aroma can be integrated with the one or more scrim layers.

At 414, the one or more melt-blown layers can be ultrasonically bonded with the one or more scrim layers. The one or more melt-blown layers can undergo ultrasonic bonding. Ultrasonic bonding can include ultrasonic welding. Ultrasonic welding can include directing high-frequency vibrations at the one or more melt-blown layers. The one or more melt-blown layers can be fused or clamped together. Ultrasonic welding can create a rapid build-up of heat that produces a weld, or bond, in a few seconds without significant melting of the one or more melt-blown layers. The aroma can be integrated with the one or more melt-blown layers during the ultrasonic bonding process. The aroma can be integrated with the one or more scrim layers during the ultrasonic bonding process.

At 416, the one or more melt-blown layers and the one or more scrim layers can be ionically charged. For example, the one or more melt-blown layers can undergo an ion charging process (e.g., ionic charging process). The one or more scrim layers can undergo an ion charging process. The aroma can be added to the one or more melt-blown layers. The aroma can be added to the one or more scrim layers. The aroma can be integrated with the one or more melt-blown layers during the ionic charging process. The aroma can be integrated with the one or more scrim layers during the ionic charging process.

At 418, the one or more melt-blown layers and the one or more scrim layers can be wound and/or doffed. For example, the one or more melt-blown layers can undergo a winding or doffing process. The aroma can be added to the one or more melt-blown layers. The one or more scrim layers can undergo a winding or doffing process. The aroma can be added to the one or more scrim layers. The aroma can be integrated with the one or more melt-blown layers during the winding or doffing process. The aroma can be integrated with the one or more scrim layers during the winding or doffing process.

In another embodiment, the method of manufacturing a face mask includes providing the face mask and adding an aroma to the face mask. Adding the aroma to the face mask can include adding a vapor aroma to the face mask. Adding the aroma to the face mask can include adding the aroma to packaging material and positioning the face mask in the packaging material such that the face mask absorbs the aroma.

In some embodiments, the face mask includes a valve (e.g., vent). For example, the face mask can include an exhalation value or a breathing valve. Adding the aroma to the face mask can include adding a scented capsule (e.g., replaceable scented capsule) into the valve. The wearer can replace the valve with different scented capsules. The scented capsules can include various aromas (e.g., menthol, lavender, banana, apple, or Aloe vera).

Each of the various embodiments disclosed herein may have any of the aspects, features, components, and configurations of the other embodiments, except where noted otherwise.

References herein to the positions of elements (e.g., “top,” “bottom,” “above,” “below,” etc.) are merely used to describe the orientation of various elements in the Figures. It should be noted that the orientation of various elements may differ according to other example embodiments, and that such variations are intended to be encompassed by the present disclosure. Further, the formation of a passage by one or more surfaces can comprise a wide variety of passage cross-sectional shapes, for example, passages having circular, rectangular, oval, etc. cross-sectional shapes.

As utilized herein, the term “substantially” and similar terms are intended to have a broad meaning in harmony with the common and accepted usage by those of ordinary skill in the art to which the subject matter of this disclosure pertains. It should be understood by those of skill in the art who review this disclosure that these terms are intended to allow a description of certain features described and claimed without restricting the scope of these features to the precise numerical ranges provided. Accordingly, these terms should be interpreted as indicating that insubstantial or inconsequential modifications or alterations of the subject matter described and claimed (e.g., within plus or minus five percent of a given angle or other value) are considered to be within the scope of the invention as recited in the appended claims. The term “approximately” when used with respect to values means plus or minus five percent of the associated value.

The terms “coupled” and the like as used herein mean the joining of two members directly or indirectly to one another. Such joining may be stationary (e.g., permanent) or moveable (e.g., removable or releasable). Such joining may be achieved with the two members or the two members and any additional intermediate members being integrally formed as a single unitary body with one another or with the two members or the two members and any additional intermediate members being attached to one another.

It is important to note that the construction and arrangement of the various example embodiments are illustrative only. Although only a few embodiments have been described in detail in this disclosure, those skilled in the art who review this disclosure will readily appreciate that many modifications to the flow structures are possible (e.g., variations in sizes, dimensions, structures, shapes and proportions of the various elements, values of parameters, mounting arrangements, use of materials, colors, orientations, etc.) without materially departing from the novel teachings and advantages of the subject matter described herein. For example, elements shown as integrally formed may be constructed of multiple parts or elements, the position of elements may be reversed or otherwise varied, and the nature or number of discrete elements or positions may be altered or varied. The order or sequence of any process or method steps may be varied or re-sequenced according to alternative embodiments. Additionally, features from particular embodiments may be combined with features from other embodiments as would be understood by one of ordinary skill in the art. Other substitutions, modifications, changes and omissions may also be made in the design, operating conditions and arrangement of the various example embodiments without departing from the scope of the present invention.

What is claimed is:

1. A face mask, comprising:
an aroma layer comprising one or more melt-blown layers, the one or more melt-blown layers comprising a plurality of fibers with an aroma integrated therewith.

2. The face mask of claim 1, wherein the aroma layer comprises an aroma layer outer side and an aroma layer inner side, the face mask further comprising:
an outer scrim layer positioned adjacent the aroma layer outer side; and
an inner scrim layer positioned adjacent the aroma layer inner side.

3. The face mask of claim 1, wherein the aroma layer comprises one or more scrim layers.

4. The face mask of claim 1, wherein the plurality of fibers comprise melt-blown polypropylene.

5. The face mask of claim 1, wherein the aroma layer comprises menthol, lavender, banana, apple, or Aloe vera.

6. The face mask of claim 1, wherein the plurality of fibers comprise an anti-fog additive. A face mask, comprising:
an aroma layer comprising one or more scrim layers, the one or more scrim layers comprising a plurality of fibers with an aroma integrated therewith.

8. The face mask of claim 7, wherein the aroma layer comprises an aroma layer outer side and an aroma layer inner side, the face mask further comprising:
an outer scrim layer positioned adjacent the aroma layer outer side; and
an inner scrim layer positioned adjacent the aroma layer inner side.

9. The face mask of claim 7, wherein the aroma layer comprises one or more melt-blown layers.

10. The face mask of claim 7, wherein the aroma layer comprises menthol, lavender, banana, apple, or Aloe vera.

11. A face mask, comprising:
a middle layer comprising one or more scrim layers and one or more melt-blown layers, the middle layer comprising a middle layer outer side and a middle layer inner side;
an outer scrim layer positioned adjacent the middle layer outer side; and
an inner scrim layer positioned adjacent the middle layer inner side;
wherein at least one of the middle layer, the outer scrim layer, or the inner scrim layer comprises a plurality of fibers with an aroma integrated therewith.

12. The face mask of claim 11, wherein at least one of the one or more melt-blown layers comprises melt-blown polypropylene.

13. The face mask of claim 11, where the least one of the middle layer, the outer scrim layer, or the inner scrim layer comprises menthol, lavender, banana, apple, or Aloe vera.

14. A method of manufacturing a face mask, the method comprising:
providing a blended mixture comprising polypropylene, a charge additive, and an aroma;
extruding the blended mixture to form melt-blown fibers;
forming an aroma layer from the melt-blown fibers;
disposing the aroma layer between an outer mask layer and an inner mask layer; and
securing the outer mask layer and the inner mask layer.

15. The method of claim 14, further comprising:
blending the polypropylene, the charge additive, and the aroma to form the blended mixture.

16. The method of claim 14, further comprising:
blending the polypropylene, the charge additive, the aroma, and an anti-fog additive to form the blended mixture.

17. The method of claim 14, wherein the aroma layer comprises menthol, lavender, banana, apple, or Aloe vera.

18. A method of manufacturing a face mask, the method comprising:
providing the face mask;
adding an aroma to the face mask.

19. The method of claim 18, wherein adding the aroma to the face mask comprises adding a vapor aroma to the face mask.

20. The method of claim 18, wherein adding the aroma to the face mask comprises:
adding the aroma to packaging material;
positioning the face mask in the packaging material such that the face mask absorbs the aroma.

21. The method of claim 18, wherein the face mask comprises a valve and wherein adding the aroma to the face mask comprises adding a scented capsule into the valve.

22. The method of claim 18, wherein the aroma comprises menthol, lavender, banana, apple, or Aloe vera.
